(19) United States
(12) Patent Application Publication
(10) Pub. No.: US 2025/0186101 A1
(43) Pub. Date: Jun. 12, 2025

(54) Steam Pop (SP) Safety Early Warning and Control System for Radio Frequency Catheter Ablation (RFCA)

(71) Applicant: Chengdu Megain Technology Co., Ltd., Chengdu (CN)

(72) Inventors: Hanxiong LIU, Chengdu (CN); Xunzhang WANG, Chengdu (CN); Yan LUO, Chengdu (CN)

(21) Appl. No.: 18/638,637

(22) Filed: Apr. 17, 2024

(30) Foreign Application Priority Data
Dec. 12, 2023 (CN) 2023116986221

(51) Int. Cl. A61B 18/14; A61B 18/00

(52) U.S. Cl. CPC A61B 18/14; A61B 2018/00577; A61B 2018/00648; A61B 2018/00702; A61B 2018/00773; A61B 2018/00791; A61B 2018/00875; A61B 2018/00898

(57) ABSTRACT

The present disclosure discloses a steam pop (SP) safety early warning and control system for radiofrequency catheter ablation (RFCA). According to the present disclosure, a new function of performing real-time early warning against an SP event and timely terminating ablation before the SP event occurs is designed. The whole system is provided with an information acquisition module, an analysis module, and a control module. The control module is configured to generate an early warning and control signal according to a main parameter combination standard, an auxiliary standard and a termination curve cluster used for terminating ablation and obtained by the analysis module, so as to achieve early warning and control of an ablation mechanism, and achieve functions of real-time early warning against SP and timely termination of ablation before the SP occurs.

### CROSS REFERENCE TO RELATED APPLICATION

This patent application claims the benefit and priority of Chinese Patent Application No. 2023116986221, filed with the China National Intellectual Property Administration on Dec. 12, 2023, the disclosure of which is incorporated by reference herein in its entirety as part of the present application.

### TECHNICAL FIELD

The present disclosure relates to the field of safety early warning and control for radiofrequency catheter ablation (RFCA), and in particular, to a steam pop (SP) safety early warning and control system for RFCA.

### BACKGROUND

RFCA, as a minimally invasive intervention method, has been recommended as a first-line treatment approach for rhythm control of atrial fibrillation (AF). Currently, RFCA of AF has entered the era of high power and short time (efficient ablation), which significantly shortens the AF ablation time, but inevitably leads to the occurrence of an SP event and other safety problems.

Physically, tissue coagulation necrosis induced by RFCA begins with the increase of temperature after 50° C., and finally turns into steam, which explodes in deep atrial myocardium. A probability of cardiac perforation, pericardial tamponade even death, or other malignant complications caused by rupture of an atrioventricular wall resulting from an SP event is much greater than that when no SP event occurs. Moreover, a probability of death caused by a non-SP event is 1/10000 or below, and a probability of death caused by an SP event is 2/100 or above. Therefore, compared with that by a non-SP event, the probability of death caused by an SP event may increase by 200 times or above. However, due to the inaudible and silent characteristics of SP events, an operator often cannot perceive SP events, and it is very difficult to estimate an increase of an SP event risk during RFCA. Therefore, taking effective measures to identify potential SP events and avoiding fatal complications whenever possible is an effective means to reduce the risk of RFCA. Up to date, there is no method that can predict whether and when an SP event will occur, to say nothing of the accurately early warning on the SP event for ablation termination in real time.

### SUMMARY

In order to solve the above problems existing in the prior art, the present disclosure provides an SP safety early warning and control system for RFCA.

To achieve the above objective, the present disclosure provides the following scheme:

The present disclosure provides a steam pop (SP) safety early warning and control system for radiofrequency catheter ablation (RFCA), including:
an information acquisition module connected to an ablation device and configured to obtain baseline information, an ablation point database of SP events in history, and ablation parameter indexes during radiofrequency ablation, where the baseline information includes: age, gender, height, and weight; the ablation parameter index includes: an ablation catheter type, an impedance curve of ablation points, impedance stability, a pressure curve, pressure stability, a temperature curve, a respiration curve, respiration stability, a heartbeat curve, heartbeat stability, an ablation time, power, a perfusion volume, an ablation index (AI) value, and anatomical characteristics of the ablation points;
an analysis module connected to the information acquisition module and configured to determine a main parameter combination standard and an auxiliary standard for terminating ablation based on ablation point data of the SP events in history, and generate a termination curve cluster corresponding to the main parameter combination standard and the auxiliary standard; and
a control module connected to the analysis module and the ablation device, configured to compare the ablation parameter indexes during the radiofrequency ablation and the termination curve cluster to generate an early warning and control signal, and configured to control an ablation mechanism in the ablation device based on the early warning and control signal.

Optionally, the information acquisition module includes:
an input unit configured to input the baseline information;
an automatic acquisition unit connected to the ablation device and configured to obtain the ablation catheter type, impedance of the ablation points, a pressure, a temperature, a respiration volume, heartbeat, the ablation time, the power, the perfusion volume, and the AI value; and
an information generating unit connected to the automatic acquisition unit and configured to determine the impedance curve of the ablation points, the impedance stability, the pressure curve, the pressure stability, the temperature curve, the respiration curve, the respiration stability, the heartbeat curve, the heartbeat stability, and the anatomical characteristics of the ablation points based on data obtained by the automatic acquisition unit, so as to obtain the ablation parameter indexes during the radiofrequency ablation.

Optionally, the analysis module includes:
a sample acquisition unit connected to the information acquisition module, and configured to obtain ablation point data of the SP events in history, and allow the ablation point data to fall into an SP+ group and a SP− group according to whether an SP event occurs during ablation;
a parameter screening unit connected to the sample acquisition unit, and configured to respectively screen distinguishing parameters of SP events from the SP+ group and the SP− group by using a statistical method;
an SP event prediction unit connected to the parameter screening unit, and configured to perform receiver operating characteristic (ROC) analysis on the distinguishing parameters of the SP events to obtain sensitivity, specificity, a negative predictive value and a positive predictive value of each distinguishing parameter for predicting SP events; and
a curve cluster generation unit connected to the SP event prediction unit, and configured to use distinguishing parameters with the sensitivity reaching 100% and the specificity reaching a set threshold as the main parameter combination standard, use other remaining distinguishing parameters as the auxiliary standard, and generate a termination curve cluster corresponding to the main parameter combination standard and the auxiliary standard.

Optionally, the main parameter combination standard includes the ablation catheter type, the impedance curve of the ablation points, the pressure curve, the temperature curve, the ablation time, a stability parameter, and the anatomical characteristics of the ablation points.

Optionally, the auxiliary standard includes the respiration curve, the respiration stability, the heartbeat curve, the heartbeat stability, and the perfusion volume.

Optionally, the control module includes:
a first relationship determining unit connected to the analysis module and configured to obtain the main parameter combination standard in the ablation parameter indexes during the radiofrequency ablation and determine a relationship between the main parameter combination standard and each curve in the termination curve cluster, where the termination curve cluster corresponding to the main parameter combination standard includes a first warning line and a second warning line;
an early warning and control signal generation unit connected to the first relationship determining unit and the ablation device, and configured to generate an ablation mechanism stop signal when the main parameter combination standard reaches the first warning line, or generate an ablation mechanism early warning signal when the main parameter combination standard reaches the second warning line, where the ablation mechanism stop signal is used to control the ablation device to stop operating; and the ablation mechanism early warning signal is used to control the ablation device to stop operating after operating for a set time;
a second relationship determining unit connected to the analysis module and configured to obtain the auxiliary standard in the ablation parameter indexes during the radiofrequency ablation and determine a relationship between the auxiliary standard and the termination curve cluster; and
a risk determining unit connected to the second relationship determining unit, and configured to retrieve the baseline information and the ablation parameter indexes during the radiofrequency ablation by the analysis module when the auxiliary standard reaches the termination curve cluster, and determine whether there is a risk of occurrence of an SP event based on the baseline information and the ablation parameter indexes during the radiofrequency ablation, where if there is the risk, the ablation mechanism stop signal is generated, or if there is no risk, the ablation mechanism is continued.

Optionally, the information acquisition module, the analysis module and the control module are integrally arranged in an upper computer or a controller; and the upper computer or the controller is connected to the ablation device.

According to specific embodiments of the present disclosure, the present disclosure has the following technical effects:

According to the present disclosure, a new function of performing real-time early warning against an SP event during operation and timely terminating ablation before the SP event occurs is designed according to laws of an ablation catheter type, an impedance curve of ablation points, impedance stability, a pressure curve, pressure stability, a temperature curve, a respiration curve, respiration stability, a heartbeat curve, heartbeat stability, anatomical characteristics of the ablation points, and other ablation parameters in radiofrequency ablation, thereby improving safety. An early warning and control signal is generated according to a main parameter combination standard, an auxiliary standard and a termination curve cluster used for terminating ablation and obtained by an analysis module, so as to achieve early warning and control of an ablation mechanism, and achieve functions of real-time early warning against SP and timely termination of ablation before the SP occurs.

### BRIEF DESCRIPTION OF THE DRAWINGS

To describe the technical solutions in embodiments of the present disclosure or in the prior art more clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without creative efforts.

FIG. 1 is a schematic structural diagram of an SP safety early warning and control system for RFCA according to the present disclosure;

FIG. 2 is a schematic diagram of an impedance curve according to the present disclosure;

FIG. 3 is a schematic diagram showing occurrence of SP according to Embodiment 1 of the present disclosure; and

FIG. 4 is a schematic diagram showing occurrence of SP according to Embodiment 2 of the present disclosure.

### DETAILED DESCRIPTION OF THE EMBODIMENTS

The technical solutions of the embodiments of the present disclosure are clearly and completely described below with reference to the accompanying drawings in the embodiments of the present disclosure. Apparently, the described embodiments are merely some rather than all of the embodiments of the present disclosure. All other embodiments obtained by those of ordinary skill in the art based on the embodiments of the present disclosure without creative efforts shall fall within the protection scope of the present disclosure.

An objective of the present disclosure is to provide an SP safety early warning and control system for RFCA, which can accurately perform early warning against an SP event and achieve real-time termination of ablation.

In order to make the above objective, features and advantages of the present disclosure clearer and more comprehensible, the present disclosure will be further described in detail below in combination with accompanying drawings and specific implementations.

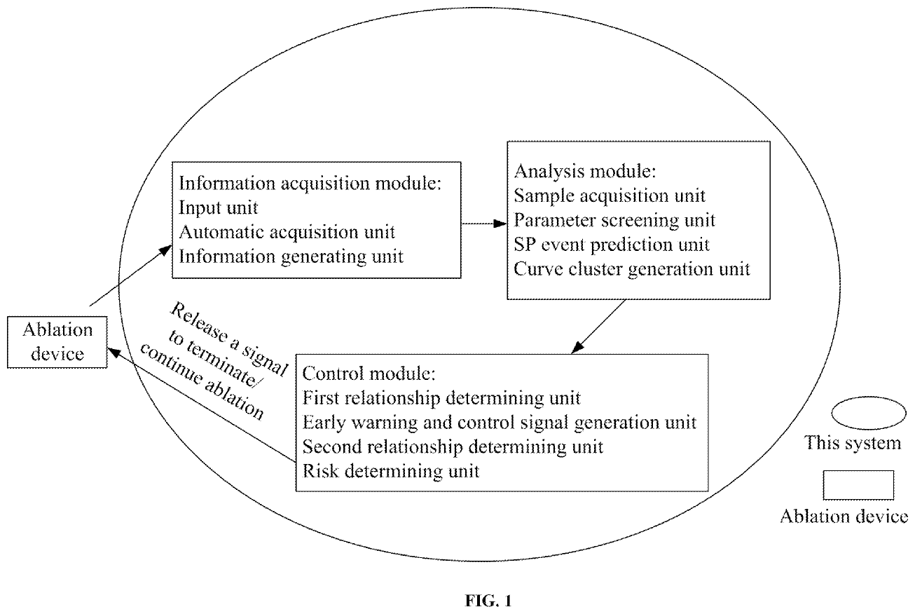

As shown in FIG. 1, an SP safety early warning and control system for RFCA according to the present disclosure includes an information acquisition module, an analysis module, and a control module. The information acquisition module is connected to the analysis module and an ablation device. Both the analysis module and the ablation device are connected to the control module.

The information acquisition module is configured to obtain baseline information, an ablation point database of SP events in history, and ablation parameter indexes during radiofrequency ablation. The baseline information includes: age, gender, height, and weight. The ablation parameter index includes: an ablation catheter type, an impedance curve of ablation points, impedance stability, a pressure curve, pressure stability, a temperature curve, a respiration curve, respiration stability, a heartbeat curve, heartbeat stability, an ablation time, power, a perfusion volume, an ablation index (AI) value, and anatomical characteristics of the ablation points. The ablation catheter type, the impedance curve of the ablation points, the impedance stability, the pressure curve, the pressure stability, the temperature curve, the respiration curve, the respiration stability, the heartbeat curve, the heartbeat stability, the ablation time, the power, the perfusion volume, the AI value, the anatomical characteristics of the ablation points, and the like are all quantifiable ablation parameters.

During practical application, the information acquisition module used in the present disclosure may include the following units:
1) an input unit configured to input the baseline information;
2) an automatic acquisition unit connected to the ablation device and configured to obtain the ablation catheter type, impedance of the ablation points, a pressure, a temperature, a respiration volume, heartbeat, the ablation time, the power, the perfusion volume, and the AI value; and
3) an information generating unit connected to the automatic acquisition unit and configured to determine the impedance curve of the ablation points, the impedance stability, the pressure curve, the pressure stability, the temperature curve, the respiration curve, the respiration stability, the heartbeat curve, the heartbeat stability, and the anatomical characteristics of the ablation points based on data obtained by the automatic acquisition unit, so as to obtain the ablation parameter indexes during the radiofrequency ablation.

Further, the analysis module is configured to determine a main parameter combination standard and an auxiliary standard for terminating ablation based on ablation point database of the SP events, and generate a termination curve cluster corresponding to the main parameter combination standard and the auxiliary standard.

During practical application, a comprehensive analysis module used in the present disclosure is configured to perform further analysis according to various baseline information and the ablation parameter indexes collected by the information acquisition module, to obtain the main parameter combination standard and the auxiliary standard for early warning against an SP event and timely termination of ablation before the SP event, and automatically generate a termination curve cluster (0 second, 1 second, 2 seconds, or the like).

For example, the analysis module used in the present disclosure is implemented by using the following units.
1) A sample acquisition unit connected to the information acquisition module, and configured to obtain the ablation point database of the SP events in history, and allow the ablation point data to fall into an SP+ group and a SP− group according to whether an SP event occurs during surgery.

For example, a process of obtaining ablation point data of SP events in history was as follows: 1,130 research objects were included, with a total of 16,955 ablation points, and SP events occurred to 26 research objects. The 26 research objects had a total of 1,576 ablation points, and SP events occurred to 32 ablation points. The collected age, gender, height, weight, and other baseline parameters (that is, the baseline information) of the 26 research objects to which SP events occurred were input into the system according to the present disclosure, and the connected ablation device automatically grabbed the ablation catheter type, the impedance curve of the ablation points, the impedance stability, the pressure curve, the pressure stability, the temperature curve, the respiration curve, the respiration stability, the heartbeat curve, the heartbeat stability, the ablation time, the power, the perfusion volume, the AI value, the anatomical characteristics of the ablation points, and all other quantifiable ablation parameter indexes of all 1,576 ablation points to which SP events occurred.

An ablation point data division process was follows: all 1,576 ablation points (including 32 ablation points with SP events) of 26 research objects to which SP events occurred fell into an SP+ group (with SP events) and an SP− group (without SP events) according to whether SP events occurred for a surgeon during radiofrequency ablation.
2) A parameter screening unit connected to the sample acquisition unit, and configured to respectively screen distinguishing parameters of SP events from the SP+ group and the SP− group by using a statistical method.
3) An SP event prediction unit connected to the parameter screening unit, and configured to perform receiver operating characteristic (ROC) analysis on the distinguishing parameters of the SP events to obtain sensitivity, specificity, a negative predictive value and a positive predictive value of each distinguishing parameter for predicting SP events.
4) A curve cluster generation unit connected to the SP event prediction unit, and configured to use distinguishing parameters with the sensitivity reaching 100% and the specificity reaching a set threshold as the main parameter combination standard, use other remaining distinguishing parameters as an auxiliary standard, and generate a termination curve cluster corresponding to the main parameter combination standard and the auxiliary standard.

During practical application, by means of a statistical method of independent sample T test or nonparametric test and chi-square test, parameters significant for distinguishing SP are selected (that is, P<0.05), including the ablation catheter type, the impedance curve, the pressure curve, a stability parameter, the temperature curve, the respiration curve, the heartbeat curve, the ablation time, the power, the anatomical characteristics of the ablation points, and the like. ROC analysis is performed on the above-mentioned parameters significant for distinguishing SP events to obtain sensitivity, specificity, a negative predictive value and a positive predictive value of each parameter for predicting SP events. The nonparametric test impedance curve, the pressure curve, the temperature curve, the respiration curve, the heartbeat curve, the ablation time, the power, the perfusion volume, the AI value, and other continuous variables are screened by using a nonparametric test method. The ablation catheter type, the ablation point impedance stability, the pressure stability, the respiration stability, the heartbeat stability, the anatomical characteristics of the ablation points and other classified variables are screened by using a chi-square test method.

Further, for the safety (the sensitivity and the negative predictive value) and effectiveness (the specificity and the positive predictive value) of radiofrequency ablation, screening a quantitative parameter combination as a new function of real-time early warning against SP and timely termination of ablation before the SP needs to meet the goal of allowing the sensitivity (negative predictive value) for predicting SP events to reach 100% and allowing the specificity to be as high as possible. The finally selected main parameter combination standard includes the catheter type, the impedance curve, the pressure curve, the temperature curve, the stability parameter, the ablation time, the anatomical characteristics of the ablation points, and the like. The parameter combination for early warning against SP has sensitivity of 100%, a negative predictive value of 100%, a positive predictive value of 22.1%, and specificity of 95.6%, that is, the safety of early warning against SP is 100% and the early warning efficiency is 95.6%.

For the parameters (such as the respiration curve, the heartbeat curve, the respiration stability, the heartbeat stability, the power, the perfusion volume, and the AI value) that fail to meet the above-mentioned parameter combination characteristics (that is, the main parameter combination standard), statistics is performed on each parameter by using a single factor Logistic regression method, and parameters that meet P<0.05 are selected, including the respiration curve, the heartbeat curve, the respiration stability, the heartbeat stability, the perfusion volume, and the like. These parameters can be used as a clinically significant auxiliary standard for early warning against SP events.

To sum up, the main parameter combination standard and the auxiliary standard for early warning against SP events can be obtained.

Still further, the control module is configured to compare the ablation parameter indexes during the radiofrequency ablation and the termination curve cluster to generate an early warning and control signal, and configured to control an ablation mechanism in the ablation device based on the early warning and control signal.

During practical application, the control module can achieve early warning and control of ablation based on the detected main parameter combination standard and the generated termination curve cluster, so as to achieve functions of real-time early warning against SP and timely termination of ablation before the SP, and it can be ensured that the sensitivity and specificity of the system for early warning against SP events are 100% and 95.6%, respectively.

During practical application, the control module can achieve its functions by arranging the following units:
1) a first relationship determining unit connected to the analysis module and configured to obtain the main parameter combination standard in the ablation parameter indexes during the radiofrequency ablation and determine a relationship between the main parameter combination standard and each curve in the termination curve cluster, where the termination curve cluster corresponding to the main parameter combination standard includes a first warning line and a second warning line;
2) an early warning and control signal generation unit connected to the first relationship determining unit and the ablation device, and configured to generate an ablation mechanism stop signal when the main parameter combination standard reaches the first warning line, or generate an ablation mechanism early warning signal when the main parameter combination standard reaches the second warning line, where the ablation mechanism stop signal is used to control the ablation device to stop operating; and the ablation mechanism early warning signal is used to control the ablation device to stop operating after operating for a set time;
3) a second relationship determining unit connected to the analysis module and configured to obtain the auxiliary standard in the ablation parameter indexes during the radiofrequency ablation and determine a relationship between the auxiliary standard and the termination curve cluster; and
4) a risk determining unit connected to the second relationship determining unit, and configured to retrieve the baseline information and the ablation parameter indexes during the radiofrequency ablation by the analysis module when the auxiliary standard reaches the termination curve cluster, and determine whether there is a risk of occurrence of an SP event based on the baseline information and the ablation parameter indexes during the radiofrequency ablation, where if there is the risk, the ablation mechanism stop signal is generated, or if there is no risk, the ablation mechanism is continued.

In the risk determining unit, the process of determining whether there is a risk of occurrence of an SP event mainly depends on medical workers and experts who perform the surgery. At this time, the medical workers and experts need to estimate a probability of occurrence of an SP event with reference to the baseline information and the ablation parameter indexes during the radiofrequency ablation. If it is determined that the probability is high, the ablation mechanism stop signal is input, or if it is found that the probability is low, the early warning is removed and the ablation mechanism is continued.

Based on the above description, the system according to the present disclosure summarizes laws of a tissue impedance curve and other ablation parameters by analyzing various baseline indexes and all quantifiable ablation parameters, and selects the quantitative parameter combination that can perform real-time early warning against SP at an early stage of ablation, that is, the main parameter combination standard mentioned above, with core parameters being the catheter type, the impedance curve, the pressure curve, the temperature curve, the stability parameter, the ablation time, and the anatomical characteristics of the ablation points.

For example, for the impedance curve parameter, the system generates an SP event curve which is fitted by points where SP events occur on the impedance curve of all ablation points, that is, a 0-second warning line for SP occurrence, and derives a collection of warning lines at any time before the occurrence of SP, that is, the warning line cluster. When a real-time impedance curve of an ablation point steadily decreases and intersects with a warning line in the warning line cluster, a mechanism of stopping ablation discharge in advance before an early warning time mapped by the warning line in this system is triggered. For example, when the impedance drops to 10 ohms after ablation for 5 seconds, reaching a −3 second warning line in the SP warning line cluster, the system automatically performs early warning for 3 seconds and then SP occurs, so that stopping of the ablation mechanism 3 seconds in advance is triggered in real time.

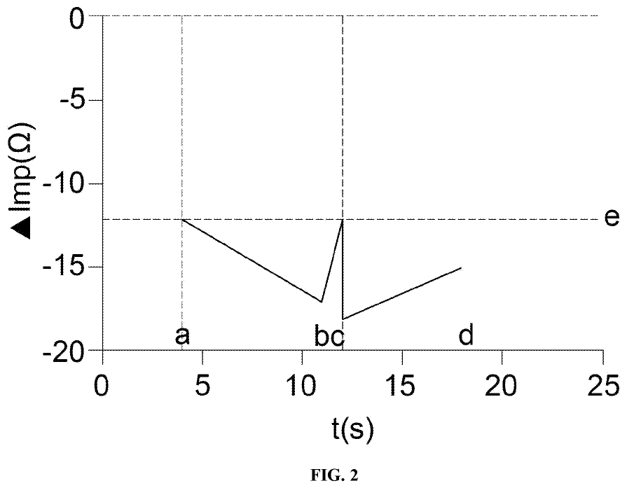

Specifically, for the impedance curve parameter, the 0-second warning line in the SP warning line cluster is shown in FIG. 2. The impedance curve shown in FIG. 2 is a curve of impedance changing with the ablation time. This curve includes but is not limited to a piecewise function in FIG. 2, which is interpreted as follows by using a formula:

$$Imp ( t ) = \{ \begin{matrix}Any value , & t ∈ [ 0 , a ] \\ K 1 t + b 1 , & t ∈ ( a , b ) \\ K 2 t + b 2 , & t ∈ [ b , c ] \\ b3 , & t = c \\ K 3 t + b 4 , & t ∈ ( c , + ∞ ]\end{matrix} ,$$

where Imp is actual radiofrequency ablation impedance (unit: ohm), t is an ablation time (unit: second) of a current target area, and K1, K2, K3, b1, b2, b3, b4, a, b and c are all constants. If t ∈[0, a], SP does not occur when Imp is any value. If t=c, an SP event inevitably occurs when Imp exceeds e. In FIG. 2, d and e are constants.

Taking the impedance curve parameter as an example, a specific operating process and achieved effects of the SP safety early warning and control system for RFCA according to the present disclosure will be described below. During actual application, for the implementation process of other parameters in the main parameter combination standard, reference may be made to the implementation process of the impedance curve in this embodiment, and details are not described herein.

First, the age, gender, height, weight and other baseline information, and the ablation catheter type, the impedance curve of the ablation points, the pressure curve, the temperature curve, the respiration curve, the heartbeat curve, the ablation time, the power, the perfusion volume, the AI value, the anatomical characteristics of the ablation points and all other quantifiable ablation parameter information of research objects are collected.

Then, the collected information is analyzed, a 0-second warning line for occurrence of SP is generated, and a warning line cluster at any time before the occurrence of SP is derived.

Finally, when a real-time impedance curve of an ablation point steadily decreases and intersects with a warning line in the warning line cluster, a mechanism of stopping ablation discharge before an early warning time mapped by the warning line is triggered, so as to control a radiofrequency ablation catheter in the ablation device to stop ablation, and achieve functions of real-time early warning against SP and timely termination of ablation before the SP occurs.

### Example 1

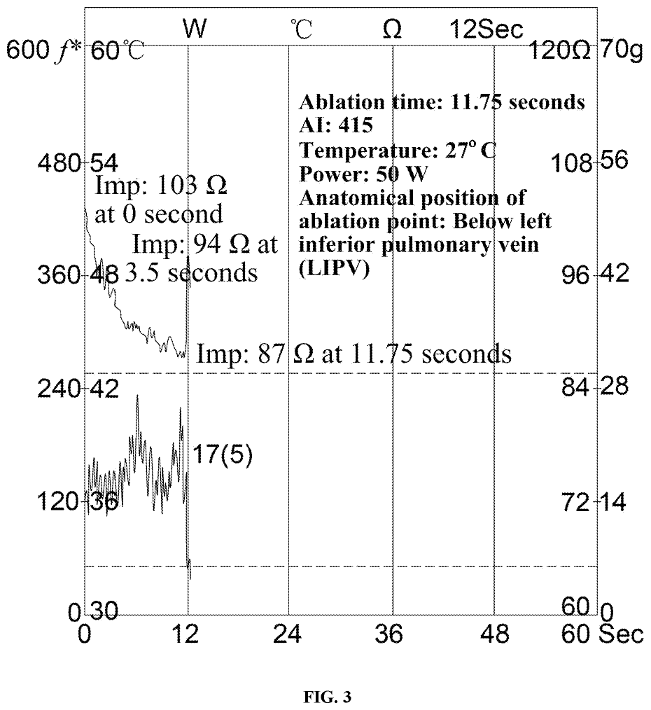

As shown in FIG. 3, when t=11.75 seconds upon occurrence of SP, a 0-second warning line of an impedance curve was touched. SP occurred when Imp steadily dropped to 16Ω. However, in fact, in this embodiment, while the 0-second warning line was generated, the system also derived a warning line cluster at any time before the SP occurred, so when the impedance curve at any time before 11.75 seconds intersected with this warning line cluster, ablation would be stopped to perform early warning against the SP in advance to ensure safety.

If t∈[0, 3.5], SP did not occur when Imp was any value; or if t=11.75 seconds, SP occurred when Imp steadily dropped to 16Ω.

In this example, although attention was paid to impedance curves of Imp and t, the curve of Imp also reflected comprehensive effects of an ablation catheter type, an impedance curve, a pressure curve, a temperature curve, a respiration curve, a heartbeat curve, an ablation time, power, a perfusion volume, an AI value, anatomical characteristics of ablation points, and all other quantifiable ablation parameters.

### Example 2

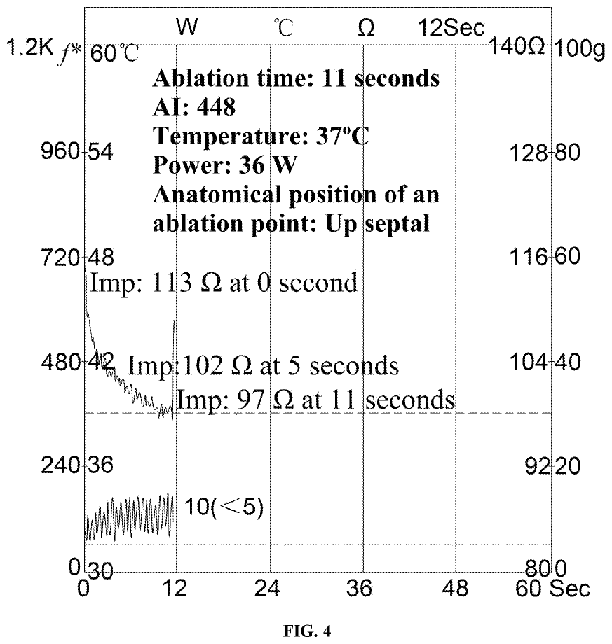

In this example, as shown in FIG. 4, when t=11 seconds, a 0-second warning line of an impedance curve was touched. SP occurred when Imp steadily dropped to 17Ω. However, in fact, in this embodiment, while the 0-second warning line was generated, the system derived a warning line cluster at any time before the SP occurred, so when the impedance curve at any time before 11 seconds intersected with this warning line cluster, ablation would be stopped, so that early warning against the SP could be performed in advance to ensure safety.

If t∈[0, 5], SP did not occur when Imp was any value; or if t=11 seconds, SP occurred when Imp steadily dropped to 17Ω.

To sum up, based on the contents described in two examples, no matter how much the impedance drops in an initial certain period of time, SP does not occur, but after a certain period of time, when the impedance steadily drops to a certain extent, the impedance will sharply rise, that is, SP will occur. However, according to the early warning and control function of this system, early warning against SP can be performed to stop ablation in advance to ensure safety.

In addition, the above-mentioned SP safety early warning and control system for RFCA according to the present disclosure can be equivalent to an upper computer or a controller, that is, the information acquisition module, the analysis module and the control module are integrally arranged in the upper computer or the controller; and the upper computer or the controller is connected to the ablation device. Based on this implementation result, a stop mechanism for early warning against an SP event can be iteratively upgraded by means of artificial Intelligence (AI) machine learning, and the like.

Embodiments of this description are described in a progressive manner, each embodiment focuses on the difference from other embodiments, and for the same and similar parts between the embodiments, reference may be made to each other.

Specific examples are used herein to explain the principles and implementations of the present disclosure. The foregoing description of the embodiments is merely intended to help understand the method of the present disclosure and its core ideas; besides, changes may be made by those of ordinary skill in the art to specific implementations and the scope of application in accordance with the ideas of the present disclosure. In conclusion, the content of this description shall not be construed as limitations to the present disclosure.

What is claimed is:

1. A steam pop (SP) safety early warning and control system for radiofrequency catheter ablation (RFCA), comprising:
an information acquisition module connected to an ablation device and configured to obtain baseline information, an ablation point database of SP events in history, and ablation parameter indexes during radiofrequency ablation, wherein the baseline information comprises: age, gender, height, and weight; the ablation parameter index comprises: an ablation catheter type, an impedance curve of ablation points, impedance stability, a pressure curve, pressure stability, a temperature curve, a respiration curve, respiration stability, a heartbeat curve, heartbeat stability, an ablation time, power, a perfusion volume, an ablation index (AI) value, and anatomical characteristics of the ablation points;
an analysis module connected to the information acquisition module and configured to determine a main parameter combination standard and an auxiliary standard for terminating ablation based on the ablation point database of the SP events in history, and generate a termination curve cluster corresponding to the main parameter combination standard and the auxiliary standard; and
a control module connected to the analysis module and the ablation device, configured to compare the ablation parameter indexes during the radiofrequency ablation and the termination curve cluster to generate an early warning and control signal, and configured to control an ablation mechanism in the ablation device based on the early warning and control signal.

2. The SP safety early warning and control system for RFCA according to claim 1, wherein the information acquisition module comprises:
an input unit configured to input the baseline information;
an automatic acquisition unit connected to the ablation device and configured to obtain the ablation catheter type, impedance of the ablation points, a pressure, a temperature, a respiration volume, heartbeat, the ablation time, the power, the perfusion volume, and the AI value; and
an information generating unit connected to the automatic acquisition unit and configured to determine the impedance curve of the ablation points, the impedance stability, the pressure curve, the pressure stability, the temperature curve, the respiration curve, the respiration stability, the heartbeat curve, the heartbeat stability, and the anatomical characteristics of the ablation points based on data obtained by the automatic acquisition unit, so as to obtain the ablation parameter indexes during the radiofrequency ablation.

3. The SP safety early warning and control system for RFCA according to claim 1, wherein the analysis module comprises:
a sample acquisition unit connected to the information acquisition module, and configured to obtain ablation point data of the SP events in history, and allow the ablation point data to fall into an SP+ group and a SP− group according to whether an SP event occurs during ablation;
a parameter screening unit connected to the sample acquisition unit, and configured to respectively screen distinguishing parameters of SP events from the SP+ group and the SP− group by using a statistical method;
an SP event prediction unit connected to the parameter screening unit, and configured to perform receiver operating characteristic (ROC) analysis on the distinguishing parameters of the SP events to obtain sensitivity, specificity, a negative predictive value and a positive predictive value of each distinguishing parameter for predicting SP events; and
a curve cluster generation unit connected to the SP event prediction unit, and configured to use distinguishing parameters with the sensitivity reaching 100% and the specificity reaching a set threshold as the main parameter combination standard, use other remaining distinguishing parameters as the auxiliary standard, and generate a termination curve cluster corresponding to the main parameter combination standard and the auxiliary standard.

4. The SP safety early warning and control system for RFCA according to claim 3, wherein the main parameter combination standard comprises the ablation catheter type, the impedance curve of the ablation points, the pressure curve, the temperature curve, the ablation time, a stability parameter, and the anatomical characteristics of the ablation points.

5. The SP safety early warning and control system for RFCA according to claim 3, wherein the auxiliary standard comprises the respiration curve, the respiration stability, the heartbeat curve, the heartbeat stability, and the perfusion volume.

6. The SP safety early warning and control system for RFCA according to claim 1, wherein the control module comprises:
a first relationship determining unit connected to the analysis module and configured to obtain the main parameter combination standard in the ablation parameter indexes during the radiofrequency ablation and determine a relationship between the main parameter combination standard and each curve in the termination curve cluster, wherein the termination curve cluster corresponding to the main parameter combination standard comprises a first warning line and a second warning line;
an early warning and control signal generation unit connected to the first relationship determining unit and the ablation device, and configured to generate an ablation mechanism stop signal when the main parameter combination standard reaches the first warning line, or generate an ablation mechanism early warning signal when the main parameter combination standard reaches the second warning line, wherein the ablation mechanism stop signal is used to control the ablation device to stop operating; and the ablation mechanism early warning signal is used to control the ablation device to stop operating after operating for a set time;
a second relationship determining unit connected to the analysis module and configured to obtain the auxiliary standard in the ablation parameter indexes during the radiofrequency ablation and determine a relationship between the auxiliary standard and the termination curve cluster; and
a risk determining unit connected to the second relationship determining unit, and configured to retrieve the baseline information and the ablation parameter indexes during the radiofrequency ablation by the analysis module when the auxiliary standard reaches the termination curve cluster, and determine whether there is a risk of occurrence of an SP event based on the baseline information and the ablation parameter indexes during the radiofrequency ablation, wherein if there is the risk, the ablation mechanism stop signal is generated, or if there is no risk, the ablation mechanism is continued.

7. The SP safety early warning and control system for RFCA according to claim 1, wherein the information acquisition module, the analysis module and the control module are integrally arranged in an upper computer or a controller; and the upper computer or the controller is connected to the ablation device.
